(19) United States
(12) Patent Application Publication
(10) Pub. No.: US 2021/0375488 A1
(43) Pub. Date: Dec. 2, 2021

(54) SYSTEM AND METHODS FOR AUTOMATIC MEDICAL KNOWLEDGE CURATION

(71) Applicant: MEDIUS HEALTH, North Sydney (AU)

(72) Inventors: Shameek GHOSH, North Sydney (AU); Budhaditya SAHA, North Sydney (AU); Suhrid SATYAL, North Sydney (AU)

(73) Assignee: MEDIUS HEALTH, North Sydney (AU)

(21) Appl. No.: 17/329,607

(22) Filed: May 25, 2021

Related U.S. Application Data
(60) Provisional application No. 63/032,401, filed on May 29, 2020.

(51) Int. Cl. G16H 70/60; G16H 70/40; G16H 70/20; G06N 20/00

(52) U.S. Cl. CPC G16H 70/60; G06N 20/00; G16H 70/20; G16H 70/40

(57) ABSTRACT

An automatic medical knowledge curation system automatically extracts medical knowledge from multiple sources, including medical journals, publications and publication databases, and stores this extracted information in the form of a large-scale medical knowledge graph. The system identifies clinical, health and life insurance risk factor entities and medical management information including disease detection, smoking, alcohol consumption patterns, lifestyle information, diagnosis, prognosis, treatment, measuring, monitoring and reporting. The system determines relationships between clinical entities using machine learning and data mining methods. The system determines relationship strengths and can also determine missing and noisy relationships.

### PRIORITY AND CROSS-REFERENCE TO RELATED APPLICATIONS

The present application claims priority to U.S. Provisional Application No. 63/032,401, entitled “System and Methods for Automatic Medical Knowledge Curation”, filed May 29, 2020, the entirety of which is hereby incorporated by reference.

### FIELD

The present invention relates generally to the field of medical knowledge curation for disease diagnosis and payer risks assessments performed by health and life insurance companies. More specifically, this invention relates to medical knowledge curation using machine learning techniques.

### BACKGROUND

Researchers have long wanted to enable quality healthcare for a broader population—especially for patients without access to medical experts. Medical expert systems allow individuals to interact with a software application that replaces a medical expert. The medical expert system typically asks questions to help diagnose symptoms and recommends further diagnostic steps or treatment. A similar line of questioning and interview is also performed by health and life insurance providers for performing health risk assessments. Medical expert systems have had limited success within narrow, specialized branches of medicine. Medical expert systems rely on medical knowledge stored in machine-friendly format, often called a knowledge base. Unfortunately, such medical expert systems require significant development effort from both medical experts and computer specialists. Medical expert systems also run the risk of quickly becoming obsolete because medical knowledge changes frequently.

Medical knowledge covers a wide range of different topics, each with different experts. Developing a primary-care expert system requires a wide range of medical knowledge which is difficult to obtain. Our medical understanding constantly changes. New diseases evolve. Researchers discover new genetic diseases every year. Treatment recommendations frequently change. Drug companies develop new drugs while diseases develop immunities to existing drugs. Our knowledge about drug effectiveness and side-effects constantly improves. In the United States, drug trials have primarily monitored men and failed to test the effects on women. A patient's sex, race and environment play a major role in medical diagnosis and treatment. For example, asthma occurs mostly in North America, Western Europe and Australia while tuberculosis occurs mostly in developing countries. In the United States, tuberculosis occurs mostly in racial and ethnic minorities.

A large-scale medical knowledge base is almost impossible to maintain using a purely manual review. People make mistakes that get introduced into the medical knowledge base. Automatic techniques are needed to verify a large-scale medical knowledge base.

Community healthcare professionals and general practitioners derive most of their knowledge of the symptoms of individual diseases from hospital-based observations. These symptoms are the most directly observable characteristics of a disease and the very basis of clinical disease classification. Medical researchers primarily distribute their findings in the form of medical papers. The medical community reviews medical papers and publishes them in medical journals. All medical professionals find it difficult to keep abreast of new medical findings, especially when the findings are published in a foreign language.

### SUMMARY

Embodiments are directed to a system that automatically processes medical text, extracts medical knowledge and updates a readily accessible, shared medical knowledge graph. These embodiments greatly benefit the medical risk assessment field. Among other benefits, such a medical knowledge graph can be used to support large-scale symptoms and risk factor analysis based on population characteristics, probabilistic diagnosis, and patient journey planning for healthcare professionals.

In accordance with one aspect, a system is disclosed that includes memory comprising a database system, wherein the database system comprises a medical knowledge graph; and a processor comprising an automatic medical knowledge curator configured to update the medical knowledge graph without human intervention by automatic extracting a plurality of clinical entities and their relationships from text data and linking the automatically extracted clinical entities to the medical knowledge graph.

The medical knowledge graph includes medical entities and relationships between those medical entities. The medical entities may include at least one selected from the group consisting of diseases, symptoms, risk factors, treatments, medications, body parts, and combinations thereof.

The medical knowledge graph and the automatic medical knowledge curator may reside in the cloud.

The system may further include a computing device comprising a medical query application in communication with the medical knowledge graph.

The text data may include a plurality of medical publications. The plurality of medical publications may be online.

The automatic medical knowledge curator may include an entity recognition module; a relationship extraction module; a relationship strength prediction module; and a noisy and missing link prediction module. The entity recognition module may generate a parsed sentences and entity list. The relationship extraction module may identify clinical entity relationships based on the parsed sentences and entity list. The relationship strength prediction module may identify a strength of the clinical entity relationships. The noisy and missing link prediction module may predict noisy and missing entity relationships.

The system may further include a machine learning classifier and wherein the automatic medical knowledge curator may use the machine learning classifier.

The automatic medical knowledge curator may use a support vector or random forest machine learning model.

In accordance with another aspect, a method is disclosed that includes automatically creating a medical knowledge graph without human intervention by: automatically extracting a plurality of clinical entities from text data; and linking the automatically extracted text data to the medical knowledge graph. The medical knowledge graph includes medical entities and relationships between those medical entities. The medical entities may include at least one selected from the group consisting of diseases, symptoms, risk factors, treatments, medications, body parts, and combinations thereof.

The method may further include receiving a query from a medical query application on a computing device in communication with the medical knowledge graph.

The text data may include a plurality of medical publications.

The method may further include generating a parsed sentences and entity list from the text data. The method may further include identifying clinical entity relationships based on the parsed sentences and entity list. The method may further include identifying a strength of the clinical entity relationships. The method may further include predicting noisy and missing entity relationships.

In accordance with a further aspect, a method is disclosed that includes training a relationship prediction machine learning model using pre-set input seed relationships; and using the model to predict an unknown relationship between multiple medical terms detected in a clinical sentence. The method may further include training a relationship weight prediction machine learning module using the pre-set input seed relationships; and using the model to predict a weight or strength of relationship of an unknown relationship between multiple medical terms detected in a clinical sentence.

In accordance with yet another aspect, a method is disclosed that includes representing nodes and links between nodes in a medical knowledge network using multi-dimensional vector embeddings; training a machine leaning model on said embeddings; and using the machine learning model to predict if an unknown link between two medical entities is a missing edge that should be flagged for a clinician or an existing link is missing or noisy. The method may further include adding new clinical entities to a knowledge base.

### BRIEF DESCRIPTION OF THE DRAWINGS

The drawings are made to point out the unique and inventive nature of the disclosed invention and to distinguish the invention from the prior art. The objects, features and advantages of the invention are detailed in the description taken together with the drawings. Within the accompanying drawings, various embodiments in accordance with the present disclosure are illustrated by way of example and not by way of limitation. It is noted that like reference numerals denote similar elements throughout the drawings.

FIG. 1 is an exemplary diagram showing a system using an automatic medical knowledge curator to update a medical knowledge graph supporting other example applications.

FIG. 2 is an exemplary diagram showing an automatic medical knowledge curator system.

FIG. 3 is a block diagram of a computing system used to implement the automatic medical knowledge curator and medical knowledge server.

FIG. 4 is an exemplary diagram showing the components of the automatic medical knowledge curator.

FIG. 5 is an exemplary flowchart showing the processing steps of the automatic medical knowledge curator.

FIG. 6 is an exemplary conceptual diagram illustrating medical entity recognition.

FIG. 7 is an exemplary diagram showing medical entity relationships in the medical knowledge graph and illustrating medical entity combination.

FIG. 8 is an exemplary diagram illustrating the creation of a medical relationship NLP training model.

FIG. 9 is an exemplary diagram illustrating the creation of a medical relationship strength training model.

FIG. 10 is an exemplary flowchart for the determination of missing and noisy medical relationship links.

### DETAILED DESCRIPTION

Reference will now be made in detail to various embodiments in accordance with the present disclosure, examples of which are illustrated in the accompanying drawings. While described in conjunction with various embodiments, it will be understood that these various embodiments are not intended to limit the present disclosure. On the contrary, the present disclosure is intended to cover alternatives, modifications and equivalents, which may be included within the scope of the present disclosure as construed according to the Claims. Furthermore, in the following detailed description of various embodiments in accordance with the present disclosure, numerous specific details are set forth in order to provide a thorough understanding of the present disclosure. However, it will be evident to one of ordinary skill in the art that the present disclosure may be practiced without these specific details or with equivalents thereof. In other instances, well known methods, procedures, components, and circuits have not been described in detail so as not to unnecessarily obscure aspects of the present disclosure.

Some portions of the detailed descriptions that follow are presented in terms of procedures, logic blocks, processing, and other symbolic representations of operations on data bits within a computer memory. These descriptions and representations are the means used by those skilled in the data processing arts to most effectively convey the substance of their work to others skilled in the art. In the present disclosure, a procedure, logic block, process, or the like, is conceived to be a self-consistent sequence of steps or instructions leading to a desired result. The steps are those utilizing physical manipulations of physical quantities. Usually, although not necessarily, these quantities take the form of electrical or magnetic signals capable of being stored, transferred, combined, compared, and otherwise manipulated in a computing system.

It should be borne in mind, however, that all of these and similar terms are to be associated with the appropriate physical quantities and are merely convenient labels applied to these quantities. Unless specifically stated otherwise as apparent from the following discussions, it is appreciated that throughout the present disclosure, discussions utilizing terms such as “implementing,” “inputting,” “operating,” “deciding,” “detecting,” “notifying,” “aggregating,” “coordinating,” “applying,” “comparing,” “engaging,” “predicting,” “recording,” “analyzing,” “determining,” “identifying,” “classifying,” “generating,” “extracting,” “receiving,” “processing,” “acquiring,” “performing,” “producing,” “providing,” “prioritizing,” “arranging,” “matching,” “measuring,” “storing,” “signaling,” “proposing,” “altering,” “creating,” “computing,” “loading,” “inferring,” or the like, refer to actions and processes of a computing system or similar electronic computing device or processor. The computing system or similar electronic computing device manipulates and transforms data represented as physical (electronic) quantities within the computing system memories, registers or other such information storage, transmission or display devices.

Various embodiments described herein may be discussed in the general context of computer-executable instructions residing on some form of computer-readable storage medium, such as program modules, executed by one or more computers or other devices. By way of example, and not limitation, computer-readable storage media may comprise non-transitory computer storage media and communication media. Generally, program modules include routines, programs, objects, components, data structures, etc., that perform particular tasks or implement particular abstract data types. The functionality of the program modules may be combined or distributed as desired in various embodiments.

Computer storage media includes volatile and nonvolatile, removable and non-removable media implemented in any method or technology for storage of information such as computer-readable instructions, data structures, program modules or other data. Computer storage media includes, but is not limited to, random access memory (RAM), read only memory (ROM), electrically erasable programmable ROM (EEPROM), flash memory or other memory technology, compact disk ROM (CD-ROM), digital versatile disks (DVDs) or other optical storage, magnetic cassettes, magnetic tape, magnetic disk storage or other magnetic storage devices, or any other medium that can be used to store the desired information and that can be accessed to retrieve that information.

Communication media can embody computer-executable instructions, data structures, and program modules, and includes any information delivery media. By way of example, and not limitation, communication media includes wired media such as a wired network or direct-wired connection, and wireless media such as acoustic, radio frequency (RF), infrared and other wireless media. Combinations of any of the above can also be included within the scope of computer-readable media.

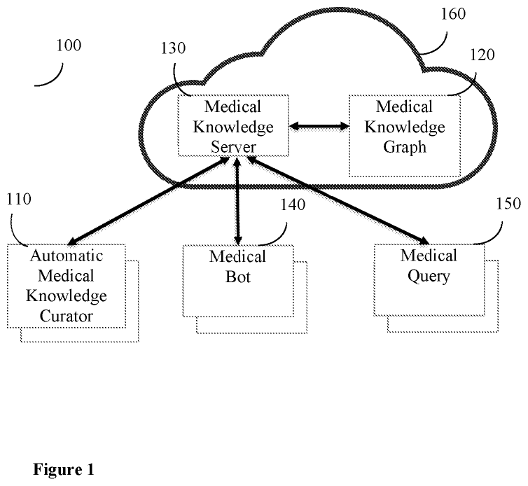

FIG. 1 is an exemplary diagram 100 showing a system using an automatic medical knowledge curator (AMKC) 110 to update a medical knowledge graph (MKG) 120 supporting other example applications 140 and 150. The MKG 120 is a knowledge base consisting of medical knowledge relating to medical entities such as diseases, symptoms, risk factors like tobacco and alcohol consumption patterns, treatments, medications. The MKG 120 also contains relationships between the medical entities such as is-a-symptom-of, is-a-type-of, is-a-risk-factor-for, is-a treatment-for. These relationships form a conceptual graph. In one embodiment, the MKG 120 resides on a persistent storage medium such as computer storage disk. In one embodiment, the MKG 120 resides in a commercial, relational database system with 3-tuple records of the form “entity-1, relationship-type, entity-2” and further records that capture properties of these relationships and medical entities. A medical knowledge server 130 resides on a computer that provides access to the MKG 120. The medical knowledge server 130 and MKG 120 reside in the cloud 160 and have a network connection that allows applications at different geographic locations to access the MKG 120. The medical knowledge server 130 and MKG 120 allow multiple applications to read from and write to the MKG 120.

In some embodiments, multiple copies of the AMKC 110 reside on separate computers that may or may not be in the cloud 160. Each copy of AMKC 110 updates the MKG 120. Multiple medical bots 140, residing on separate computing devices, communicate with the medical knowledge server 130 and read data from the MKG 120. Each medical bot acts as an expert system and can be to used to give health care advice. Multiple medical query applications 150, residing on separate computing devices, communicate with the medical knowledge server 130 and read data from the MKG 120. Each medical query application allows someone to access information in the MKG 120. For example, a doctor might form a query asking for the symptoms of a specific disease. A medical researcher might ask which medical papers have indicated a specific medical relationship.

The AMKC 110 can be used in other configurations to that shown in FIG. 1. The AMKC can interact directly with an MKG stored locally without going through a medical knowledge server. Other applications besides medical bots and a medical query system can take advantage of the MKG.

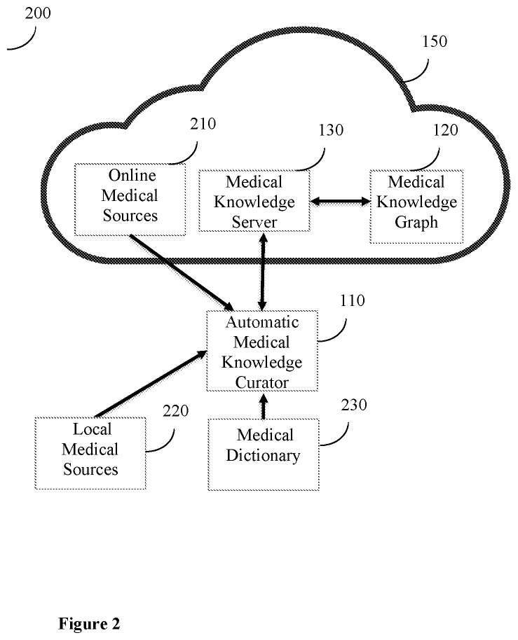

FIG. 2 is an exemplary diagram showing an automated medical knowledge curator system 200 containing an automated medical knowledge curator application (AMKC) 110. The AMKC 110 searches for and reads online medical sources 210 stored in the cloud 150 using a network connection. The medical sources 210 include medical papers, medical journals, medical blogs, medical databases, medical forum discussions and other online medical publications. Exemplary medical journals and publications that are accessed include the Journal of American Medical Association (JAMA), British medical journal (BMJ), EMedicine and MedicineNet. Medical databases include PubMed providing research publication access, the Online Mendelian Inheritance in Man (OMIM) database providing a catalogue of genetic disorders, PubMed Central (PMC), the US National Library of Medicine's digital archive of life sciences journal literature, and the NCBI Web site providing a database of home pages available for reference. It will be appreciated that additional or alternative online medical publications may also be accessed. The AMKC 110 also reads local medical sources 220 from a computer-storage device. Local medical sources include medical documents stored locally on a computer disk and paper medical documents which are scanned and converted to readable text using optical character recognition. Medical sources include prose, tables and graphs. The AMKC 110 can automatically scan the internet looking for new or updated medical information. The AMKC 110 performs internet keyword searches to identify possible medical sources and then sends the results to a system operator or automatically determines if the search result identified a suitable medical source. The AMKC 110 can periodically check for new issues of known medical publications by examining the content of known medical publication web-sites. The AMKC 110 can also process specific medical publications specified by a system operator. For example, a system operator may restrict the input to credible medical sources.

The AMKC 110 reads a list of medical entity names defined in a medical dictionary 230 from a computer-storage device. The AMKC 110 uses the medical dictionary 230 to identify medical entities mentioned in medical sources 210 and 220. In one embodiment, the medical dictionary 230 is based on the publicly available Unified Medical Language System (UMLS). UMLS is a set of files and software that brings together many health and biomedical vocabularies and standards to enable interoperability between computer systems. The UMLS includes the Metathesaurus, a large biomedical thesaurus organized by concept, or meaning, that links similar names for the same concept from nearly 200 different vocabularies. The Metathesaurus also identifies useful relationships between concepts and preserves the meanings, concept names, and relationships from each vocabulary.

After reading medical sources 210 and 220 and the medical dictionary 230, the AMKC 110 updates the MKG 120 by sending network messages to the medical knowledge server 130. The AMKC may read the medical dictionary 230 using software procedures associated with medical dictionary 230, by using one or more database queries, by requesting data over a network and by reading from a storage medium. When updating the MKG 120, the AMKC 110 stores a medical source reference so that MKG users can identify the original medical knowledge source.

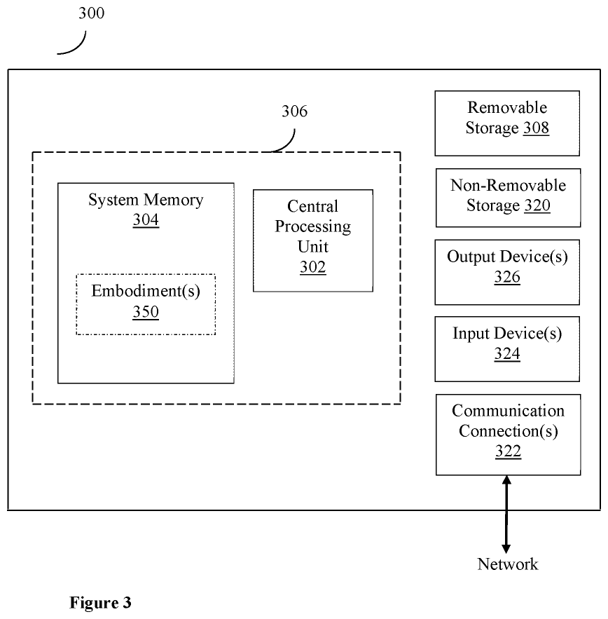

FIG. 3 is a block diagram of an example of a computing system 300 upon which one or more various embodiments described herein may be implemented in accordance with various embodiments of the present disclosure. In a basic configuration, the system 300 includes at least one processing unit 302 and memory 304. This basic configuration is illustrated in FIG. 3 by dashed line 306. The system 300 may also have additional features and/or functionality. For example, the system 300 may also include additional storage (e.g., removable and/or non-removable) including, but not limited to, magnetic or optical disks or tape. Such additional storage is illustrated in FIG. 3 by removable storage 308 and non-removable storage 320.

The system 300 may also contain communications connection(s) 322 that allow the device to communicate with other devices, e.g., in a networked environment using logical connections to one or more remote computers. Furthermore, the system 300 may also include input device(s) 324 such as, but not limited to, a voice input device, touch input device, keyboard, mouse, pen, touch input display device, etc. In addition, the system 300 may also include output device(s) 326 such as, but not limited to, a display device, speakers, printer, etc.

In the example of FIG. 3, the memory 304 includes computer-readable instructions, data structures, program modules, and the like associated with one or more various embodiments 350 in accordance with the present disclosure. Embodiments 350 include the AMKC computer-readable instructions and data and the medical knowledge server computer-readable instructions and data. However, the embodiment(s) 350 may instead reside in any one of the computer storage media used by the system 300 or may be distributed over some combination of the computer storage media or may be distributed over some combination of networked computers but is not limited to such.

It is noted that the computing system 300 may not include all of the elements illustrated by FIG. 3. Moreover, the computing system 300 can be implemented to include one or more elements not illustrated by FIG. 3. It will be appreciated that the computing system 300 can be utilized or implemented in any manner similar to that described and/or shown by the present disclosure but is not limited to such.

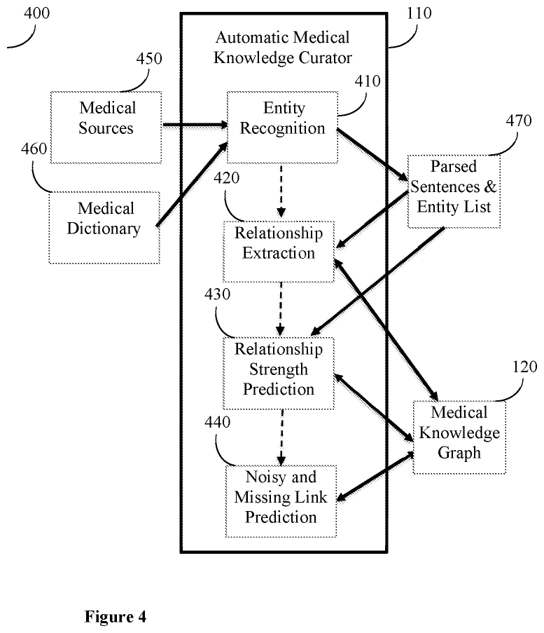

FIG. 4 is an exemplary diagram 400 showing the components of the AMKC 110. The AMKC 110 has four modules: entity recognition 410, relationship extraction 420, relationship strength prediction 430 and noisy and missing link prediction 440. In one embodiment, these four modules operate sequentially. In a second embodiment, the four modules operate in parallel, in a pipe-lined manner; for example, entity recognition 410 could work on a second medical paper while relationship extraction 420 works on the data from a first medical paper.

Entity recognition module 410 reads medical sources 450 and medical dictionary 460. Entity recognition module 410 identifies medical entities in medical sources 450 where those medical entities are defined by medical dictionary 460. Terms in the text are used as input for string-similarity matching against the names in the medical dictionary 460 and closest matches are assigned, indexed and marked with their semantic type as per the medical dictionary entity type. The entity recognition module 410 produces parsed sentences and entity list 470 which may be stored in memory, story on a disk, or forwarded as network packages.

Relationship extraction module 420 reads the parsed sentences and entity list 470 and the MKG 120. Relationship extraction module 420 identifies medical entity relationships and updates the MKG 120.

Relationship strength prediction module 430 reads the MKG 120, identifies the strength of medical entity relationships, and updates the MKG 120.

Noisy and missing link prediction module 440 reads the MKG 120, predicts noisy and missing entity relationships and updates the MKG 120.

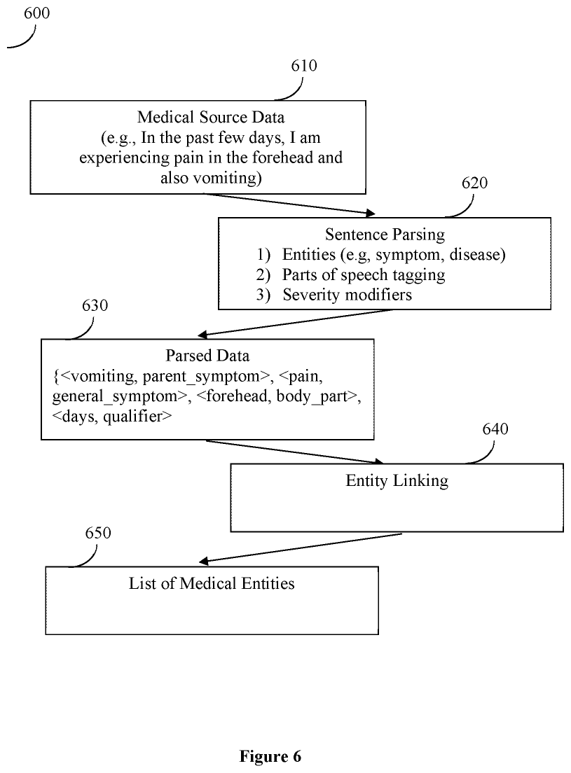

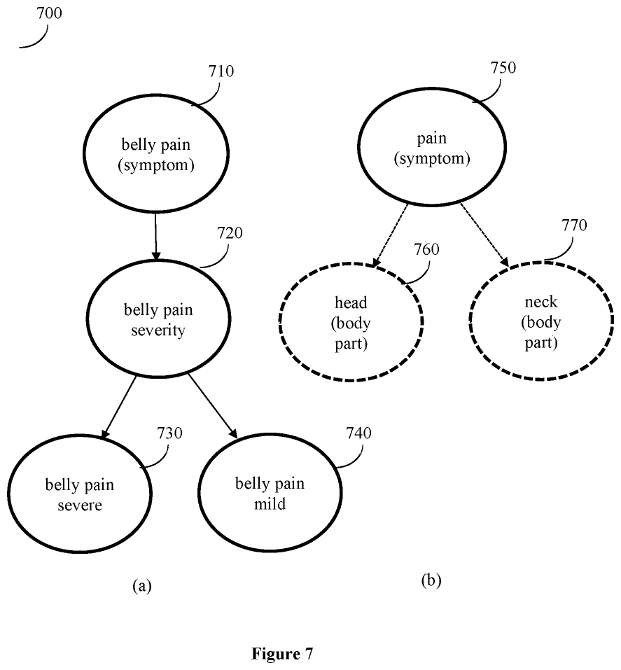

Additional details about the entity recognition module 410, relationship extraction module 420, relationship strength prediction module 430 and the noisy and missing link prediction module are discussed below and in particular with respect to FIGS. 6 and 7.

In one embodiment, entity recognition 410 and relationship extraction 420 operate on a single medical paper at a time. In other embodiments, entity recognition 410 and relationship extraction 420 operate on parts of a medical paper or on multiple medical papers at a time. In one embodiment, relationship strength prediction 430 and noisy and missing link prediction 440 operate on the entire MKG 120 after processing each medical paper. In other embodiments, relationship strength prediction 430 and noisy and missing link prediction 440 operate on a relevant subset of MKG 120 after processing multiple medical papers.

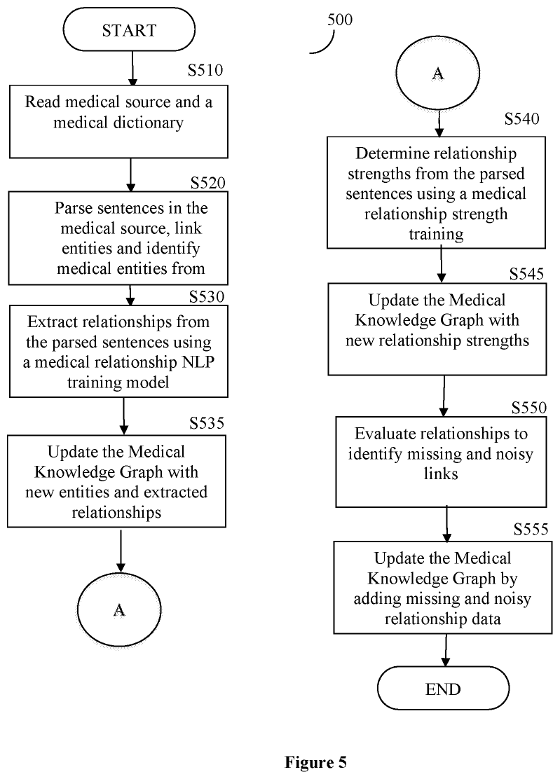

FIG. 5 is an exemplary flowchart 500 showing a process performed by the automatic medical knowledge curator (AMKC). In step S510, the AMKC reads medical source and medical dictionary data from a computer-storage device.

In step S520, the AMKC parses the medical source data, identifying medical entities defined in the medical dictionary. The AMKC produces parsed sentence data by searching for end-of-sentence delimiters and medical entities in the medical source data. Although the AMKC parses one sentence at a time, this does not prevent the AMKC from linking references between different sentences. The AMKC combines identified terms together to form specific medical entities as further described with respect, in particular, to FIG. 7.

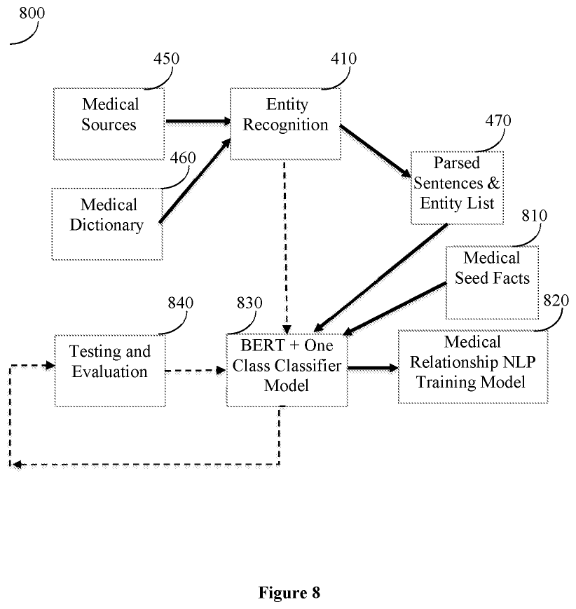

In step S530, the AMKC processes the parsed sentence data and extracts medical relationships between the medical entities using one or more medical relationships, natural language parsing (NLP) training models as further described with respect, in particular, to FIG. 8. In one embodiment, the AMKC uses multiple NLP training models with one training model for each medical relationship type such as is-a-symptom-of, is-a-type-of, is-a-risk-factor-for, is-a treatment-for, etc. In a second embodiment, the AMKC use one or more NLP training models that can detect multiple medical relationship types. The AMKC can support multiple natural languages (e.g., English, Bengali and Hindi) using multiple NLP training models or a combination of NLP training models. At step S535, the AMKC updates the MKG by adding new medical entities and relationships.

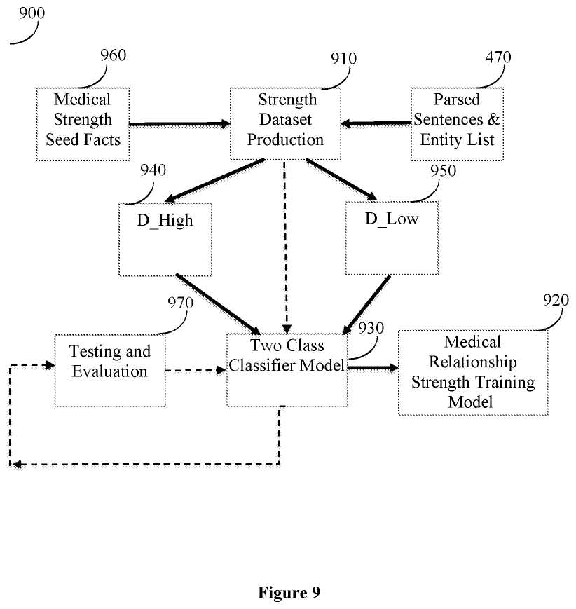

In step S540, the AMKC determines relationship strengths using a medical relationship strength training model. The strength represents the likelihood that a medical relationship exists and is determined from the parsed sentence data as further described with respect, in particular, to FIG. 9. At step S545, the AMKC updates the MKG by adding new medical relationship strengths.

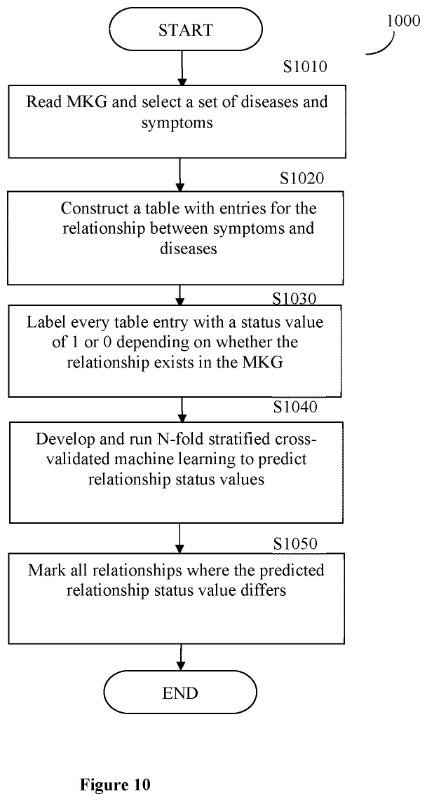

In step S550, the AMKC identifies missing and noisy medical relationship links using a combination of training models as further described with respect, in particular, to FIG. 10. A missing medical relationship is one that has not been reported but which the training models predict. Similarly, a noisy medical relationship is one that has been reported but which the training models do not predict. Missing and noisy medical relationship links can be a good indicator of the need for medical research. At step S555, the AMKC updates the MKG by adding missing and noisy medical relationship data.

FIG. 6 is an exemplary conceptual diagram 600 illustrating medical entity recognition 410. In this example, the medical source data 610 contains a sentence saying “In the past few days, I am experiencing pain in the forehead and also vomiting”. Sentence parsing 620 parses the sentence 610 producing parsed data 630. In general, the sentence parsing 620 identifies medical entities such as symptoms (e.g. “fever”, “belly pain”) and diseases (e.g. “flu”, “Gastritis”), tags parts of speech, and identifies severity modifiers. The sentence parsing 620 identifies qualifiers including: name of body part (e.g. “chest”, “abdomen”), name of body fluid, time information (minutes, days, months), and events (present, past). The primary, identified keywords (e.g. headache, fever) are augmented with qualifiers (e.g. dull, days) to identify fine-grained symptoms like “headache, days to months”, “fever, severe”, “belly pain, the right side of the abdomen”.

In the example of FIG. 6, the parsed data 630 lists entities (e.g., vomiting) and each entity's respective type (e.g., parent symptom). The entity linking process module 640 combines entities, as illustrated in FIG. 7 and discussed below, and then produces a list of medical entities 650.

FIG. 7 is an exemplary diagram 700 showing medical entity relationships and illustrating medical entity combination. FIG. 7(a) shows nodes within the MKG. FIG. 7(b) shows nodes during entity recognition 410. Nodes 710-740 illustrate a typical part of the MKG. Node 710 represents the belly pain symptom. Node 720 represents belly pain severity and the connecting arrow indicates it is a property of the belly pain symptom. Nodes “belly pain severe” 730 and “belly pain mild” 740 are types of belly pain severity 720. Nodes 750-770 illustrate the combination of entities. Node 750 represents the pain symptom. Nodes head 760 and neck 770 are qualifiers of pain and drawn with dotted borders to indicate temporary nodes that won't be stored in the MKG. The medical dictionary defines “pain”, “head pain” and “neck pain” as medical symptoms and “head” and “neck” as body parts. The AMKC recognizes that the combination of medical entity “pain” and its body part qualifier “head” together form the known medical entity “head pain”. The AMKC combines the concepts of head and pain to form the known medical entity of “head pain”.

FIG. 8 is an exemplary diagram 800 illustrating the creation of a medical relationship NLP training model 820. Medical professionals manually create a set of medical seed facts 810 and select medical sources 450 related to those facts. The entity recognition module 410 reads medical sources 450 and medical dictionary 460. Entity recognition 410 identifies medical entities in medical sources 450 where those medical entities are defined by medical dictionary 460. Entity recognition 410 produces parsed sentences and entity list 470.

The system reads medical seed facts 810 and extracts all medical source sentences where a medical seed fact occurs. A medical seed fact has a format such as “(Disease A, has_symptom, Symptom K)”. Here, the seed fact is encoded as a triple—(A, relationship, K). All sentences where A and K have occurred in the same sentence are data mined. This process is repeated for each seed fact in medical seed facts 810. At this point, the system has generated a large dataset D′ of extracted sentences that should match each seed fact in medical seed facts 810.

The system trains a machine learning, one class classifier model 830 on D′ (where D′ is the training dataset). The features used to construct the machine learning model consist of the contextual terms, their correlations, frequency, and discriminative word patterns. Bidirectional Encoder Representations from Transformers (BERT) is a known technique for NLP pre-training. In one embodiment, the system uses BERT during training. The output machine learning model is the medical relationship NLP training model 820 that can be used on any new parsed sentences and entity list 470 in the future. The system will typically employ testing and evaluation 840 before using the medical relationship NLP training model 820 in a production system. In one embodiment, system operators evaluate results and modify the medical seed facts 810, selected medical sources 450 and training methods. For example, if the medical sources 450 don't provide extracted sentences matching every seed fact, the system operator adds medical sources that do.

FIG. 9 is an exemplary diagram 900 illustrating the creation of a medical relationship strength training model 920. Medical professionals manually create a set of medical strength seed facts 960 and select medical sources related to those facts. Medical strength seed facts 960 are like medical seed facts 810 with added information about relationship strength. As in the description of FIG. 8, the system parses medical sources and produces parsed sentences and entity list 470. The system employs a strength dataset production module 910 to generate training datasets 940 and 950. The strength dataset production module 910 first generates a dataset D′ of extracted sentences that match seed facts in medical strength seed facts 960 and then allocates sentences to either D_High 940 or D_Low 950. Training dataset D_high 940 represents the subset of D′ where the corresponding seed fact indicated high strength and training dataset D_low 930 represents the subset of D′ where the corresponding seed fact indicated low strength. The system trains a machine learning, two class classifier model 930 on the two training datasets 940 and 950, producing medical relationship strength training model 920. The system will typically employ testing and evaluation 970 before using the medical relationship strength training model 920 in a production system.

The example of FIG. 9 shows a two-class classifier model. Other embodiments may use a multi-class classifier model to distinguish more strength categories. The medical strength seed facts 960 may similarly employ two strength categories or may employ multiple strength categories.

FIG. 10 is an exemplary flowchart for the determination of missing and noisy medical relationship links 1000. The example of FIG. 10 discusses missing and noisy links between symptoms and diseases but the same method is applied to other types of medical relationships.

In step S1010, the AMKC reads the MKG and selects a set of diseases and symptoms. The AMKC may select all diseases and symptoms in the MKG, or may select a related subset that have been recently updated.

In step S1020, the AMKC constructs a table with entries for combination of symptom and disease. Table 1 gives an example of such a table.

TABLE 1
Combinations of Symptoms and Diseases

| | | Feature Vector | |
|---|---|---|---|
| | | (Disease Vector. | Relationship |
| Disease | Symptom | SymptomVector) | Status |
| Asthma | Cough | [1.01, 1.12. . . . ] | 1 |
| Common Cold | Cough | [0.81, 1.22, . . . ] | 1 |
| Asthma | Knee pain | [0.91, 1.45, . . . ] | 0 |
| . . . | . . . | . . . | . . . |

The first two columns list possible diseases and symptoms. The third column lists a feature vector suitable for machine learning. The combinations of disease and symptoms represent nodes in a conceptual hypergraph and are known as an embedding space. The feature vector represents a multi-dimensional vector embedding of the relationships within the MKG. The feature vector indicates proximity between nodes in the conceptual hypergraph defined by the MKG. There are many ways of constructing the feature vectors. In one embodiment, the AMKC uses the node2vec framework available on open source site Github. Other embodiments may use other encoding such as DeepWalk or LINE. The node2vec framework learns low-dimensional representations for nodes in a graph by optimizing a neighborhood preserving objective. The objective is flexible, and the algorithm accommodates for various definitions of network neighborhoods by simulating biased random walks.

In step S1030, the AMKC fills in the fourth column of Table 1 listing the relationship status. A value of 1 indicates a known relationship present in the MKG. A value of 0 indicates no such relationship. The relationship status values are called class labels in machine learning terminology.

In step S1040, the AMKC runs a N-fold stratified cross validation to predict relationship status values. Typical values of N are 3, 4 and 5 which can give similar results. The AMKC splits the table values into N sets where each set has an equal percentage of class labels. The stratification helps balance the label distribution between the splits and the cross validation ensures that the entire dataset is used for machine learning and prediction (without overlap between the training and prediction sets). The AMKC fits a machine learning classifier on the feature vectors, representing disease-symptoms properties and the relationship status, class labels. The AMKC first removes one of the datasets and trains the classifier on the remaining N−1 datasets. The AMKC now uses the training model to predict class labels for the excluded set. The AMKC repeats this training and prediction N times so that all the N sets have their class labels predicted once. Multiple machine learning classifier models are possible. In one embodiment, the AMKC uses a support vector machine. In machine learning, support-vector machines (SVMs, also support-vector networks) are supervised learning models with associated learning algorithms that analyze data used for classification and regression analysis. Given a set of training examples, each marked as belonging to one or the other of two categories, an SVM training algorithm builds a model that assigns new examples to one category or the other, making it a non-probabilistic binary linear classifier (although methods such as Platt scaling exist to use SVM in a probabilistic classification setting). An SVM model is a representation of the examples as points in space, mapped so that the examples of the separate categories are divided by a clear gap that is as wide as possible. New examples are then mapped into that same space and predicted to belong to a category based on the side of the gap on which they fall. The gamma hyperparameter for the SVM is used to control the decision boundary of the classifier. In a second embodiment, the AMKC uses a random forest machine learning model. Random forests or random decision forests are an ensemble learning method for classification, regression and other tasks that operate by constructing a multitude of decision trees at training time and outputting the class that is the mode of the classes (classification) or mean prediction (regression) of the individual trees.

In step S1050, the AMKC updates the MKG by marking all relationships where the predicted relationship status differs from the existing value. The AMKC stores a property value associated with each of the relationships. A system operator may ask qualified medical personnel to check these marked relationships or may accept the changes automatically.

The inventors believe this is the first time that a system has been able to automatically determine missing and noisy medical relationships. A large-scale medical knowledge base (knowledge graph) is almost impossible to maintain using a purely manual review. People make mistakes that get introduced into the medical knowledge base. Automatically determining missing and noisy medical relationships is essential in the development of large-scale medical knowledge bases. The clinical missing link and noise correction method reduces the manual data review process for clinicians and also predicts potential relationships that exist between a disease and a symptom in the medical knowledge graph, reducing errors.

The medical knowledge base is an important component for various medical tasks like symptom checking, differential diagnosis prediction, clinical decision making, and medication recommendations. Since a medical knowledge base may contain thousands of clinical entities with tens of thousands of links, the manual curation of the medical knowledge base would require an extensive level of human efforts and there would be errors. Embodiments of the invention are able to detect noisy data and missing edges in the medical knowledge base, resulting in improvements of the performance of the model by 13 to 30% on accuracy. The accurate prediction of noisy links and missing links in a knowledge graph greatly improves the operational performance of a clinical review process during the construction of the medical knowledge base.

All examples and conditional language recited herein are intended for pedagogical purposes to aid the reader in understanding the principles of the present disclosure and the concepts contributed by the inventor to furthering the art and are to be construed as being without limitation to such specifically recited examples and conditions. Moreover, all statements herein reciting principles, aspects, and embodiments of the present disclosure, as well as specific examples thereof, are intended to encompass both structural and functional equivalents thereof. Additionally, it is intended that such equivalents include both currently known equivalents as well as equivalents developed in the future, e.g., any elements developed that perform the same function, regardless of structure.

The foregoing descriptions of various specific embodiments in accordance with the present disclosure have been presented for purposes of illustration and description. They are not intended to be exhaustive or to limit the present disclosure to the precise forms disclosed, and many modifications and variations are possible in light of the above teaching. The present disclosure is to be construed according to the Claims and their equivalents.

What is claimed is:

1. A system comprising:
memory comprising a database system, wherein the database system comprises a medical knowledge graph; and
a processor comprising an automatic medical knowledge curator configured to update the medical knowledge graph without human intervention by:
automatically extracting a plurality of clinical entities from text data from a plurality of medical publications using a medical dictionary; and
linking the automatically extracted clinical entities to the medical knowledge graph.

2. The system of claim 1, wherein the processor uses the medical dictionary to identify known clinical entities prior to automatically extracting the plurality of clinical entities from the text data from the plurality of medical publications.

3. The system of claim 2, wherein the medical knowledge graph comprises at least one selected from the group consisting of diseases, symptoms, risk factors, treatments, medications, body parts and combinations thereof.

4. The system of claim 3, wherein the medical knowledge graph comprises relationships between the plurality of clinical entities.

5. The system of claim 1, wherein the medical knowledge graph and the automatic medical knowledge curator reside in the cloud.

6. The system of claim 1, further comprising:
a computing device comprising a medical query application in communication with the medical knowledge graph.

7. The system of claim 1, wherein the plurality of medical publications are online.

8. The system of claim 1, wherein the automatic medical knowledge curator comprises:
an entity recognition module;
a relationship extraction module;
a relationship strength module; and
a noisy and missing link prediction module.

9. The system of claim 8, wherein the entity recognition module generates a parsed sentences and entity list.

10. The system of claim 9, wherein the relationship extraction module identifies clinical entity relationships based on the parsed sentences and entity list.

11. The system of claim 10, wherein the relationship strength prediction module identifies a strength of the clinical entity relationships.

12. The system of claim 11, wherein the noisy and missing link prediction module predicts noisy and missing entity relationships.

13. The system of claim 1, further comprising a machine learning classifier and wherein the automatic medical knowledge curator uses the machine learning classifier.

14. The system of claim 1, wherein the automatic medical knowledge curator uses one of a support vector or a random forest machine learning model.

15. A method comprising:
automatically creating a medical knowledge graph without human intervention by:
automatically extracting a plurality of clinical entities from text data from a plurality of medical publications using a medical dictionary; and
linking the automatically extracted text data to the medical knowledge graph.

16. The method of claim 15, wherein the medical knowledge graph comprises at least one selected from the group consisting of diseases, symptoms, risk factors, treatments, medications, body parts, and combinations thereof.

17. The method of claim 16, wherein the medical knowledge graph comprises relationships between the plurality of clinical entities.

18. The method of claim 15, further comprising receiving a query from a medical query application on a computing device in communication with the medical knowledge graph.

19. The method of claim 15, further comprising generating a parsed sentences and entity list from the text data.

20. The method of claim 19, further comprising identifying clinical entity relationships based on the parsed sentences and entity list.

21. The method of claim 20, further comprising identifying a strength of the clinical entity relationships.

22. The method of claim 21, further comprising predicting noisy and missing entity relationships.

23. A method comprising:
training a relationship prediction machine learning model using pre-set input seed relationships; and
using the model to extract a plurality of clinical entity relationships from text data from a plurality of medical publications using a medical dictionary.

24. The method of claim 23 further comprising:
training a relationship weight prediction machine learning module using the pre-set input seed relationships; and
using the model to determine a weight or strength of the plurality of clinical entity relationships.

25. A method comprising:
representing nodes and links between nodes in a medical knowledge network using multi-dimensional vector embeddings;
training a machine leaning model on said embeddings; and
using the machine learning model to predict if an unknown link between two medical entities is a missing edge that should be flagged for a clinician or an existing link is missing or noisy.

26. The method of claim 25, further comprising adding new clinical entities to a knowledge graph.
